(19) United States
(12) Patent Application Publication
(10) Pub. No.: US 2007/0142450 A1
(43) Pub. Date: Jun. 21, 2007

(54) Novel urea derivatives and their medical use

(76) Inventors: Bjarne H. Dahl, Ballerup (DK); Dan Peters, Ballerup (DK); Gunnar M. Olsen, Ballerup (DK); Tino Dyhring Jorgensen, Ballerup (DK); Daniel B. Timmermann, Ballerup (DK)

Correspondence Address: BIRCH STEWART KOLASCH & BIRCH, PO BOX 747, FALLS CHURCH, VA, 22040-0747, US

(21) Appl. No.: 10/590,104

(22) PCT Filed: Mar. 16, 2005
(86) PCT No.: PCT/EP05/51183

§ 371 Date: Aug. 22, 2006

Related U.S. Application Data
(60) Provisional application No. 60/557,698, filed on Mar. 31, 2004.

(30) Foreign Application Priority Data
Mar. 29, 2004 (DK) PA 2004 00498

(51) Int. Cl. A61K 31/4015; A61K 31/40; C07D 207/40; C07D 207/04

(52) U.S. Cl. 514/408; 514/427; 548/577; 548/545

(57) ABSTRACT

This invention relates to novel urea derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel urea derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides urea derivatives of Formula I

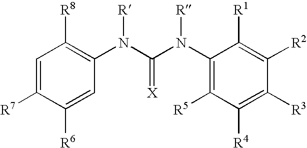

any of its enantiomers or any mixture of its enantiomers, or a prodrug, or a pharmaceutically-acceptable addition salt thereof, wherein

X represents O, S or NR′″; wherein R′″ represents hydrogen, alkyl, cycloalkyl, cycloalkyl-alkyl or cyano;

R′ and R″, independently of each other, represent hydrogen, alkyl, cycloalkyl or cycloalkyl-alkyl;

R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein R″″ and R′″″, independently of each other, represent hydrogen, alkyl, cycloalkyl, cycloalkyl-alkyl, alkenyl, phenyl or benzyl; and Ar represents an aryl group or an aromatic mono- or polycyclic heterocyclic group; or R¹ represents a group of formula —CONR″″R′″″ or —SO₂—NR″″R′″″, wherein R″″ and R′″″, independently of each other, represent hydrogen, alkyl, cycloalkyl, cycloalkyl-alkyl, alkenyl, phenyl or benzyl; or R″″ and R′″″ together with the nitrogen atom to which they are attached form a heterocyclic ring; or

R¹ represents a group of formula

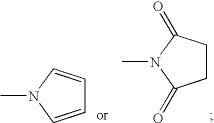

R² represents hydrogen, hydroxy, halo, haloalkyl, haloalkoxy, cyano, nitro or amino;

R³ represents hydrogen, hydroxy, halo, haloalkyl, haloalkoxy, cyano, nitro or amino;

R⁴ represents hydrogen, alkyl, hydroxy, halo, haloalkyl, haloalkoxy, cyano, nitro or amino;

R⁵ represents hydrogen, hydroxy, halo, haloalkyl, haloalkoxy, cyano, nitro or amino;

R⁶ represents hydrogen, hydroxy, halo, haloalkyl, haloalkoxy, cyano, nitro, amino or phenyl;

R⁷ represents hydrogen, hydroxy, halo, haloalkyl, haloalkoxy, cyano, nitro, amino or phenyl; and

R⁸ represents hydrogen, hydroxy, halo, haloalkyl, haloalkoxy, alkoxy, cyano, nitro or amino.

In a second aspect the invention provides pharmaceutical compositions comprising a therapeutically effective amount of the urea derivative of the invention, or a pharmaceutically-acceptable addition salt thereof, together with at least one pharmaceutically-acceptable carrier or diluent.

Viewed from another aspect the invention relates to the use of the urea derivative of the invention, or a pharmaceutically-acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the urea derivative of the invention.

Other objects of the invention will be apparent to the person skilled in the art from the following detailed description and examples.

### DETAILED DISCLOSURE OF THE INVENTION

Urea Derivatives

In its first aspect the invention provides urea derivatives of Formula I

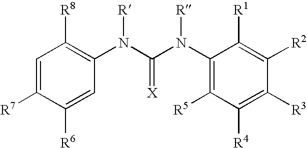

any of its enantiomers or any mixture of its enantiomers, or a prodrug, or a pharmaceutically-acceptable addition salt thereof, wherein

X represents O, S or NR′″; wherein R′″ represents hydrogen, alkyl, cycloalkyl, cycloalkyl-alkyl or cyano;

R′ and R″, independently of each other, represent hydrogen, alkyl, cycloalkyl or cycloalkyl-alkyl;

R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein R″″ and R′″″, independently of each other, represent hydrogen, alkyl, cycloalkyl, cycloalkyl-alkyl, alkenyl, phenyl or benzyl; and Ar represents an aryl group or an aromatic mono- or polycyclic heterocyclic group; or R¹ represents a group of formula —CONR″″R′″″ or —SO₂—NR″″R′″″, wherein R″″ and R′″″, independently of each other, represent hydrogen, alkyl, cycloalkyl, cycloalkyl-alkyl, alkenyl, phenyl or benzyl; or R″″ and R″″ together with the nitrogen atom to which they are attached form a heterocyclic ring; or

R¹ represents a group of formula

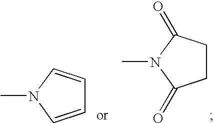

R² represents hydrogen, hydroxy, halo, haloalkyl, haloalkoxy, cyano, nitro or amino;

R³ represents hydrogen, hydroxy, halo, haloalkyl, haloalkoxy, cyano, nitro or amino;

R⁴ represents hydrogen, alkyl, hydroxy, halo, haloalkyl, haloalkoxy, cyano, nitro or amino;

R⁵ represents hydrogen, hydroxy, halo, haloalkyl, haloalkoxy, cyano, nitro or amino;

R⁵ represents hydrogen, hydroxy, halo, haloalkyl, haloalkoxy, cyano, nitro, amino or phenyl;

R⁷ represents hydrogen, hydroxy, halo, haloalkyl, haloalkoxy, cyano, nitro, amino or phenyl; and

R⁸ represents hydrogen, hydroxy, halo, haloalkyl, haloalkoxy, alkoxy, cyano, nitro or amino.

In a preferred embodiment the urea derivative of the invention is a compound of Formula I, wherein X represents O, S or NR′″; wherein R′″ represents hydrogen, alkyl, cycloalkyl, cycloalkyl-alkyl or cyano.

In a more preferred embodiment X represents O.

In another preferred embodiment the urea derivative of the invention is a compound of Formula I, wherein R′ and R″, independently of each other, represent hydrogen, alkyl, cycloalkyl or cycloalkyl-alkyl.

In a more preferred embodiment both of R′ and R″ represent hydrogen.

In a third preferred embodiment the urea derivative of the invention is a compound of Formula I, wherein R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein R″″ and R′″″, independently of each other, represent hydrogen, alkyl, cycloalkyl, cycloalkyl-alkyl, alkenyl, phenyl or benzyl; and Ar represents an aryl group or an aromatic mono- or polycyclic heterocyclic group; or R¹ represents a group of formula —CONR″″R′″″ or —SO₂—NR″″R′″″, wherein R″″ and R′″″, independently of each other, represent hydrogen, alkyl, cycloalkyl, cycloalkyl-alkyl, alkenyl, phenyl or benzyl; or R″″ and R′″″ together with the nitrogen atom to which they are attached form a heterocyclic ring; or R¹ represents a group of formula

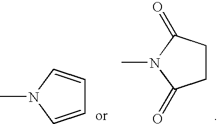

In a more preferred embodiment R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein R″″ and R′″″, independently of each other, represent hydrogen, alkyl, cycloalkyl, cycloalkyl-alkyl, alkenyl, phenyl or benzyl; and Ar represents phenyl, pyrrolyl, imidazolyl, pyrazolyl or pyridinyl; or R¹ represents a group of formula —CONR″″R′″″ or —SO₂—NR″″R′″″, wherein R″″ and R′″″, independently of each other, represent hydrogen, alkyl, cycloalkyl, cycloalkyl-alkyl, alkenyl, phenyl or benzyl; or R″″ and R′″″ together with the nitrogen atom to which they are attached form a heterocyclic ring selected from pyrrolidinyl, piperidinyl, morpholinyl and piperazinyl.

In an even more preferred embodiment R¹ represents hydrogen, alkyl, hydroxy, alkoxy, amino, or a group of formula —NR″″(CO)R′″″; wherein R″″ and R′″″, independently of each other, represent hydrogen, alkyl, cycloalkyl, cycloalkyl-alkyl, alkenyl, phenyl or benzyl; or R¹ represents a group of formula

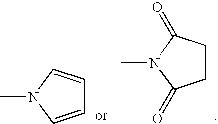

In a still more preferred embodiment R¹ represents hydrogen, alkyl, hydroxy, alkoxy, amino or —NH(CO)alkyl.

In a yet more preferred embodiment R¹ represents hydrogen, methyl, hydroxy, methoxy, amino or —NH(CO)methyl.

In a yet still more preferred embodiment R¹ represents hydrogen, hydroxy, amino or —NH(CO)methyl.

In a fourth preferred embodiment the urea derivative of the invention is a compound of Formula I, wherein R² represents hydrogen, hydroxy, halo, haloalkyl, haloalkoxy, cyano, nitro or amino.

In a more preferred embodiment R² represents hydrogen, hydroxy or halo.

In an even more preferred embodiment R² represents hydrogen, hydroxy, Cl or Br.

In a fifth preferred embodiment the urea derivative of the invention is a compound of Formula I, wherein R³ represents hydrogen, hydroxy, halo, haloalkyl, haloalkoxy, cyano, nitro or amino.

In a more preferred embodiment R³ represents hydrogen, hydroxy, halo or nitro.

In an even more preferred embodiment R³ represents hydrogen, hydroxy or halo.

In a sixth preferred embodiment the urea derivative of the invention is a compound of Formula I, wherein

R⁴ represents hydrogen, alkyl, hydroxy, halo, haloalkyl, haloalkoxy, cyano, nitro or amino.

In a more preferred embodiment R⁴ represents hydrogen, alkyl or halo.

In an even more preferred embodiment R⁴ represents hydrogen, methyl or Cl.

In a seventh preferred embodiment the urea derivative of the invention is a compound of Formula I, wherein R⁵ represents hydrogen, hydroxy, halo, haloalkyl, haloalkoxy, cyano, nitro or amino.

In a more preferred embodiment R⁵ represents hydrogen, nitro or amino.

In an even more preferred embodiment R⁵ represents hydrogen or amino.

In an eighth preferred embodiment the urea derivative of the invention is a compound of Formula I, wherein R⁶ represents hydrogen, hydroxy, halo, haloalkyl, haloalkoxy, cyano, nitro, amino or phenyl.

In a more preferred embodiment R⁶ represents hydrogen, hydroxy, halo, haloalkyl, haloalkoxy, cyano, nitro or amino.

In an even more preferred embodiment R⁶ represents hydrogen, halo, haloalkyl or phenyl.

In a still more preferred embodiment R⁶ represents hydrogen, haloalkyl or phenyl.

In a yet more preferred embodiment R⁶ represents hydrogen, halo or haloalkyl.

In a ninth preferred embodiment the urea derivative of the invention is a compound of Formula I, wherein R⁷ represents hydrogen, hydroxy, halo, haloalkyl, haloalkoxy, cyano, nitro, amino or phenyl.

In a more preferred embodiment R⁷ represents hydrogen, nitro or phenyl.

In an even more preferred embodiment R⁷ represents hydrogen or phenyl.

In a still more preferred embodiment R⁷ represents hydrogen or nitro.

In a tenth preferred embodiment the urea derivative of the invention is a compound of Formula I, wherein R⁸ represents hydrogen, hydroxy, halo, haloalkyl, haloalkoxy, alkoxy, cyano, nitro or amino.

In a more preferred embodiment R⁸ represents hydrogen, hydroxy, halo or alkoxy.

In a more preferred embodiment R⁸ represents hydrogen or halo.

In an eleventh preferred embodiment the urea derivative of the invention is a compound of Formula I, wherein

R¹ represents hydrogen, alkyl, hydroxy, alkoxy, amino or —NH(CO)methyl;

R² represents hydrogen, hydroxy or halo;

R³ represents hydrogen, hydroxy, halo or nitro;

R⁴ represents hydrogen, alkyl or halo;

R⁵ represents hydrogen, alkyl, amino or nitro;

R⁶ represents hydrogen, halo, haloalkyl or phenyl;

R⁷ represents hydrogen or phenyl; and

R⁸ represents hydrogen, hydroxy, halo or alkoxy.

In a more preferred embodiment

R¹ represents hydrogen, hydroxy, amino or N-alkylcarbonyl-amino;

R² represents hydrogen, hydroxy, chloro or bromo;

R³ represents hydrogen, hydroxy, chloro or nitro;

R⁴ represents hydrogen, methyl or chloro;

R⁵ represents hydrogen, amino or nitro;

R⁶ represents hydrogen, chloro or trifluoromethyl;

R⁷ represents hydrogen or nitro; and

R⁸ represents hydrogen, hydroxy, chloro or methoxy.

In another more preferred embodiment

R¹ represents hydrogen, methyl, hydroxy, alkoxy, amino or N-methylcarbonyl-amino;

R² represents hydrogen, hydroxy, chloro or bromo;

R³ represents hydrogen, hydroxy, chloro or nitro;

R⁴ represents hydrogen, methyl or chloro;

R⁵ represents hydrogen, methyl, amino or nitro;

R⁶ represents hydrogen, chloro, trifluoromethyl or phenyl;

R⁷ represents hydrogen or phenyl; and

R⁸ represents hydrogen, hydroxy, chloro or methoxy.

In a twelfth preferred embodiment the urea derivative of the invention is a compound of Formula I, wherein

R¹ represents hydroxy;

R² represents hydrogen or halo;

R³ represents hydrogen or nitro;

R⁴ represents hydrogen or halo;

R⁵ represents hydrogen, nitro or amino;

R⁶ represents halo or haloalkyl;

R⁷ represents hydrogen or phenyl; and

R⁸ represents hydrogen, halo or alkoxy.

In a more preferred embodiment

R¹ represents hydroxy;

R² represents hydrogen or halo;

R³ represents hydrogen or nitro;

R⁴ represents halo;

R⁵ represents hydrogen or nitro;

R⁶ represents halo or haloalkyl;

R⁷ represents hydrogen; and

R⁸ represents hydrogen, halo or alkoxy.

In another more preferred embodiment

R¹ represents hydroxy;

R² represents hydrogen, chloro or bromo;

R³ represents hydrogen or nitro;

R⁴ represents hydrogen or chloro;

R⁵ represents hydrogen, nitro or amino;

R⁶ represents chloro or trifluoromethyl;

R⁷ represents hydrogen or phenyl; and

R⁶ represents hydrogen, chloro, hydroxy or methoxy.

In a thirteenth preferred embodiment the urea derivative of the invention is a compound of Formula I, wherein

R¹ represents hydrogen;

R² represents hydrogen, hydroxy or halo;

R³ represents hydrogen or hydroxy;

R⁴ represents alkyl or halo;

R⁵ represents hydrogen;

R⁶ represents hydrogen, haloalkyl or phenyl;

R⁷ represents hydrogen or phenyl; and

R⁸ represents hydrogen or halo.

In a more preferred embodiment

R¹ represents hydrogen;

R² represents hydrogen, hydroxy or halo;

R³ represents hydrogen or hydroxy;

R⁴ represents alkyl or halo;

R⁵ represents hydrogen;

R⁶ represents haloalkyl;

R⁷ represents hydrogen; and

R⁸ represents hydrogen, hydroxy, halo or alkoxy.

In an even more preferred embodiment

R² represents hydrogen, hydroxy or chloro; and

R⁶ represents trifluoromethyl.

In a fourteenth preferred embodiment the urea derivative of the invention is a compound of Formula I, wherein

R¹ represents alkyl, alkoxy, amino or N-alkylcarbonyl-amino;

R² represents hydrogen;

R³ represents hydroxy or halo;

R⁴ represents hydrogen or halo;

R⁵ represents hydrogen;

R⁶ represents haloalkyl;

R⁷ represents hydrogen; and

R⁸ represents hydrogen or halo.

In a fifteenth preferred embodiment the urea derivative of the invention is a compound of Formula I, wherein

R¹ represents hydrogen or hydroxy;

R² represents hydrogen or hydroxy;

R³ represents hydrogen;

R⁴ represents hydrogen, alkyl or halo;

R⁵ represents hydrogen or amino;

R⁶ represents hydrogen or haloalkyl;

R⁷ represents hydrogen or nitro; and

R⁸ represents hydrogen or hydroxy.

In a sixteenth preferred embodiment the urea derivative of the invention is a compound of Formula I, wherein

R¹ represents hydrogen, amino or N-alkylcarbonyl-amino;

R² represents hydrogen;

R³ represents hydroxy or halo;

R⁴ represents halo;

R⁵ represents hydrogen;

R⁶ represents haloalkyl;

R⁷ represents hydrogen; and

R⁸ represents hydrogen or halo.

In a seventeenth preferred embodiment the urea derivative of the invention is a compound of Formula I, wherein

R¹ represents hydrogen, amino or —NH(CO)methyl;

R² represents hydrogen;

R³ represents hydroxy or chloro;

R⁴ represents chloro;

R⁵ represents hydrogen;

R⁶ represents trifluoromethyl;

R⁷ represents hydrogen; and

R⁸ represents hydrogen or chloro.

In a most preferred embodiment the urea derivative of Formula I is

N-(3-Chloro-6-hydroxy-phenyl)-N′(2-chloro-5-trifluoromethyl-phenyl)-urea;

N-(2-Amino-6-hydroxy-phenyl)-N′-(3-trifluoromethyl-phenyl)-urea;

N-(5-Chloro-2-hydroxy-phenyl)-N′-(2-hydroxy-4-nitro-phenyl)-urea;

N-(2-Amino-4,5-dichloro-phenyl)-N′-(2-chloro-5-trifluoromethyl-phenyl)-urea;

N-{4,5-Dichloro-2-[3-(2-chloro-5-trifluoromethyl-phenyl)-ureido]phenyl}-acetamide;

N-(3-Chloro-4-hydroxy-phenyl)-N′-(3-trifluoromethyl-phenyl)-urea;

N-(4-Hydroxy-6-methyl-phenyl)-N′-(3-trifluoromethyl-phenyl)-urea;

N-(3,5-Dichloro-4-hydroxy-phenyl)-N′-(3-trifluoromethyl-phenyl)urea;

N-(2-Chloro-5-trifluoromethyl-phenyl)-N′-(3,5-dichloro-4-hydroxy-phenyl)urea;

N-(Biphenyl-3-yl)-N′-(3,5-dichloro-4-hydroxy-phenyl)urea;

N-(Biphenyl-4-yl)-N′-(3,5-dichloro-4-hydroxy-phenyl)urea;

N-(Biphenyl-4-yl)-N′-(5-chloro-2-hydroxy-phenyl)urea;

N-(3,5-Dichloro-2-hydroxy-phenyl)-N′-(2-chloro-5-trifluoromethyl-phenyl)-urea;

N-(3-Bromo-5-chloro-2-hydroxy-phenyl)-N′-(2-chloro-5-trifluoromethyl-phenyl)-urea;

N-(2-Chloro-5-trifluoromethyl-phenyl)-N′-(3-hydroxy-5-methyl-phenyl)urea;

N-(3-Hydroxy-5-methyl-phenyl)-N′-(3-trifluoromethyl-phenyl)urea;

N-(2-Chloro-5-trifluoromethyl-phenyl)-N′-(4-hydroxy-2-methyl-phenyl)urea;

N-(5-Chloro-2-methoxy-phenyl)-N′-(2-chloro-5-trifluoromethyl-phenyl)urea;

N-(2-Hydroxy-6-nitro-phenyl)-N′-(3-trifluoromethyl-phenyl)urea; or

N-(3-Chloro-6-methoxy-phenyl)-N′-(2-hydroxy-4-nitro-phenyl)urea;

or an enantiomer or a mixture of its enantiomers, or a pharmaceutically-acceptable addition salt thereof.

Any combination of two or more of the embodiments described herein is considered within the scope of the present invention.

Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention an alkenyl group designates a straight or branched carbon chain containing one or more double bonds, including di-enes, tri-enes and poly-enes. In a preferred embodiment the alkenyl group of the invention comprises of from two to eight carbon atoms (C₂₋₈-alkenyl), more preferred of from two to six carbon atoms (C₂₋₆-alkenyl), including at least one double bond. In a most preferred embodiment the alkenyl group of the invention is ethenyl; 1- or 2-propenyl (allyl); 1-, 2- or 3-butenyl, or 1,3-butdienyl; 1-, 2-, 3-, 4- or 5-hexenyl, or 1,3-hexdienyl, or 1,3,5-hextrienyl; 1-, 2-, 3-, 4-, 5-, 6-, or 7-octenyl, or 1,3-octdienyl, or 1,3,5-octtrienyl, or 1,3,5,7-octtetraenyl.

In the context of this invention an alkoxy group designates an “alkyl-O—” group, wherein alkyl is as defined above. Examples of preferred alkoxy groups of the invention include methoxy and ethoxy.

In the context of this invention halo represents fluoro, chloro, bromo or iodo, and haloalkyl group designates an alkyl group as defined herein, which alkyl group is substituted one or more times with halo. Thus a trihalomethyl group represents e.g. a trifluoromethyl group, a trichloromethyl group, and similar trihalo-substituted methyl groups. Preferred haloalkyl groups of the invention include trihalomethyl, preferably —CF₃.

In the context of this invention a haloalkoxy group designates an alkoxy group as defined herein, which alkoxy group is substituted one or more times with halo. Preferred haloalkoxy groups of the invention include trihalomethoxy, preferably —OCF₃.

In the context of this invention an aryl group designates a monocyclic or polycyclic aromatic hydrocarbon group. Examples of preferred aryl groups of the invention include phenyl, indenyl, naphthyl, azulenyl, fluorenyl, and anthracenyl. In a most preferred embodiment an aryl group of the invention is phenyl.

In the context of this invention an aromatic mono- or polycyclic heterocyclic group is an aromatic mono-, bi- or polycyclic compound, which holds one or more heteroatoms in its ring structure. The term “bi- and poly-heterocyclic groups” includes benzo-fused five- and six-membered heterocyclic rings containing one or more heteroatoms. Preferred heteroatoms include nitrogen (N), oxygen (O), and sulphur (S).

Preferred aromatic mono-heterocyclic groups of the invention include pyrrolyl, imidazolyl, pyrazolyl and pyridinyl.

In the context of this invention a heterocyclic ring formed by R″″ and R′″″ together with the nitrogen atom to which they are attached designates a monocyclic heterocyclic ring including at least one N-atom and optionally one or two additional heteroatoms selected from N, S and O. Preferred heterocyclic rings include pyrrolidinyl, piperidinyl, morpholinyl and piperazinyl.

Pharmaceutically Acceptable Salts

The urea derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts, such as the sodium salt of a chemical compound of the invention containing a carboxy group.

Steric Isomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers Racemates and Resolutions”, John Wiley and Sons, New York (1981).

Optical active compounds can also be prepared from optical active starting materials.

Methods of Producing Urea Derivatives

The urea derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the compounds of the invention are used for the treatment of diseases, disorders, or conditions relating to the central nervous system. Such diseases or disorders includes anxiety, cognitive disorders, learning deficit, memory deficits and dysfunction, Alzheimer's disease, attention deficit, attention deficit hyperactivity disorder (ADHD), Parkinson's disease, Huntington's disease, Amyotrophic Lateral Sclerosis, Gilles de la Tourette's syndrome, psychosis, depression, mania, manic depression, schizophrenia, obsessive compulsive disorders (OCD), panic disorders, eating disorders such as anorexia nervosa, bulimia and obesity, narcolepsy, nociception, AIDS-dementia, senile dementia, periferic neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, and jet-lag.

In a preferred embodiment diseases, disorders, or conditions relating to the central nervous system for which the compounds of the invention are used are cognitive disorders, psychosis, schizophrenia and/or depression.

In another preferred embodiment the compounds of the invention may be useful for the treatment of diseases, disorders, or conditions associated with smooth muscle contractions, including convulsive disorders, angina pectoris, premature labour, convulsions, diarrhoea, asthma, epilepsy, tardive dyskinesia, hyperkinesia, premature ejaculation, and erectile difficulty.

In yet another preferred embodiment the compounds of the invention may be useful for the treatment of endocrine disorders, such as thyrotoxicosis, pheochromocytoma, hypertension and arrhythmias.

In still another preferred embodiment the compounds of the invention may be useful for the treatment of neurodegenerative disorders, including transient anoxia and induced neuro-degeneration.

In even another preferred embodiment the compounds of the invention may be useful for the treatment of inflammatory diseases, disorders, or conditions, including inflammatory skin disorders such as acne and rosacea, Chron's disease, inflammatory bowel disease, ulcerative colitis, and diarrhoea.

In still another preferred embodiment the compounds of the invention may be useful for the treatment of mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to post therapeutic neuralgia, or to peripheral nerve injury.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

In another aspect, the compounds of the invention are used as diagnostic agents, e.g. for the identification and localisation of nicotinic receptors in various tissues.

Pharmaceutical Compositions

In another aspect the invention provides novel pharmaceutical compositions comprising a therapeutically effective amount of urea derivative of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the urea derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

The urea derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a urea derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### BRIEF DESCRIPTION OF THE DRAWING

The present invention is further illustrated by reference to the accompanying drawing, in which:

FIG. 1 shows the effect of a compound of the invention (Compound 1) in a Morris Water Maze study of hippocampal-dependent learning and memory performance in combination with Scopolamine (SCO), a reference muscarinic antagonist [Latency measured in seconds (s), in four trials per day (∘ Vehicle+Vehicle; ●Vehicle+0.1 mg Scopolamine; Δ 10 mg/kg of Compound 1+0.1 mg of Scopolamine; ▴ 30 mg/kg of Compound 1+0.1 mg/kg of Scopolamine), for four consecutive days (Day 1; Day 2; Day 3; Day 4)].

### EXAMPLES

The invention is further illustrated with reference to the following examples, which are not intended to be in any way limiting to the scope of the invention as claimed.

### Preparatory Examples 1-6

### Example 1

### N-(3-Chloro-6-hydroxy-phenyl)-N′-(2-chloro-5-trifluoromethyl-phenyl)urea (Compound 1A)

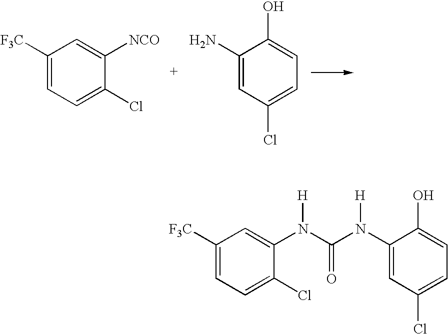

20 g of 2-chloro-5-trifluoromethyl-phenyl isocyanate (90 mmol) and 13 g of 5-chloro-2-hydroxy-aniline (90 mmol) in 600 mL of toluene under nitrogen atmosphere was stirred for 90 minutes. The precipitate was isolated by filtration, washed with cool toluene and dissolved in 150 mL of acetone. The solution was poured into 300 mL of water with 3 mL of 4 M hydrochloric acid and the product was isolated by filtration and dried under a heat lamp. Yield 30 g (91%). Mp. 172-173° C.

The following compounds were prepared in analogy herewith:

N-(3-Chloro-6-methoxy-phenyl)-N′-(2-hydroxy-4-nitro-phenyl)urea (Compound 1B); Mp. 225-226° C.;

N-(2-Hydroxy-6-nitro-phenyl)-N′-(3-trifluoromethyl-phenyl)urea (Compound 1C); Mp. 174-175° C.;

N-(2-Amino-4,5-dichloro-phenyl)-N′-(2-chloro-5-trifluoromethyl-phenyl)urea (Compound 1D); Mp. 207-209° C.;

N-(3-Chloro-4-hydroxy-phenyl)-N′-(3-trifluoromethyl-phenyl)urea (Compound 1E); Mp. 194-196° C.;

N-(4-Hydroxy-6-methyl-phenyl)-N′-(3-trifluoromethyl-phenyl)urea (Compound 1F); Mp. 185-186° C.;

N-(3,5-Dichloro-4-hydroxy-phenyl)-N′-(3-trifluoromethyl-phenyl)urea (Compound 1G); Mp. 215-218° C.;

N-(2-Chloro-5-trifluoromethyl-phenyl)-N′-(3,5-dichloro-4-hydroxy-phenyl)urea (Compound 1H); Mp. 213-21 5° C.;

N-(Biphenyl-3-yl)-N′-(3,5-dichloro-4-hydroxy-phenyl)urea (Compound 1I); Mp. 252-253° C.;

N-(Biphenyl-4-yl)-N′-(3,5-dichloro-4-hydroxy-phenyl)urea (Compound 1J); Mp. 249-251° C.;

N-(Biphenyl-4-yl)-N′-(5-chloro-2-hydroxy-phenyl)urea (Compound 1K); Mp. 191-192° C.;

N-(2-Chloro-5-trifluoromethyl-phenyl)-N′-(3-hydroxy-5-methyl-phenyl)urea (Compound 1L); Mp. 190-192° C.;

N-(3-Hydroxy-5-methyl-phenyl)-N′-(3-trifluoromethyl-phenyl)urea (Compound 1M); Mp. 188-190° C.;

N-(2-Chloro-5-trifluoromethyl-phenyl)-N′-(4-hydroxy-2-methyl-phenyl)urea (Compound 1N); Mp. 210-211° C.; and

N-(5-Chloro-2-methoxy-phenyl)-N′-(2-chloro-5-trifluoromethyl-phenyl)urea (Compound 1O); Mp. 172-176° C.

### Example 2

### N-(3,5-Dichloro-6-hydroxy-phenyl)-N′-(2-chloro-trifluoromethyl-phenyl)urea (Compound 2A)

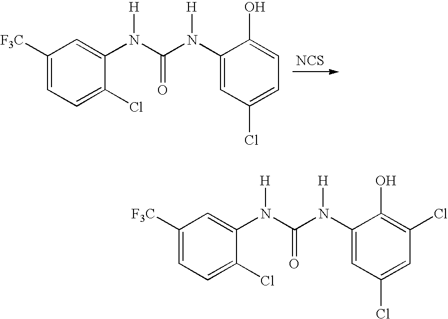

1 g of N-(3-chloro-6-hydroxy-phenyl)-N′-(2-chloro-5-trifluoromethyl-phenyl)urea (2.7 mmol) in 50 mL of acetonitrile was cooled to −20° C. and added 0.4 g of N-chlorosuccinimide. The reaction mixture was stirred at −20° C. for 30 minutes, then at room temperature for 16 hours. The reaction mixture was poured into 100 mL of water, the precipitate was isolated by filtration, washed with water and dried under a heat lamp. Yield 1 g (93%). Mp. 161-163° C.

### Example 3

### N-(3-Bromo-5-chloro-2-hydroxy-phenyl)-N′-(2-chloro-5-trifluoromethyl-phenyl)urea (Compound 3A)

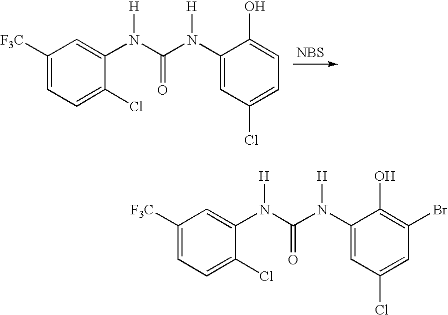

1 g of N-(3-chloro-6-hydroxy-phenyl)-N′-(2-chloro-5-trifluoromethyl-phenyl)urea (2.7 mmol) and 0.5 g of N-bromosuccinimide (2.7 mmol) in 30 mL of acetonitrile was stirred at room temperature for 2 hours, added 50 mg of N-bromosuccinimide and stirred for 16 hours. The reaction mixture was poured into 75 mL of water, the precipitate was isolated by filtration, washed with water and dried under a heat lamp. The precipitate was dissolved in acetone and filtrated. The filtrate was poured into water, the precipitate isolated by filtration and dried under a heat lamp. Yield 0.7 g (58%). Mp. 261-274° C.

### Example 4

### N-(3-Chloro-6-hydroxy-phenyl)-N′-(2-hydroxy-4-nitro-phenyl)urea (Compound 4A)

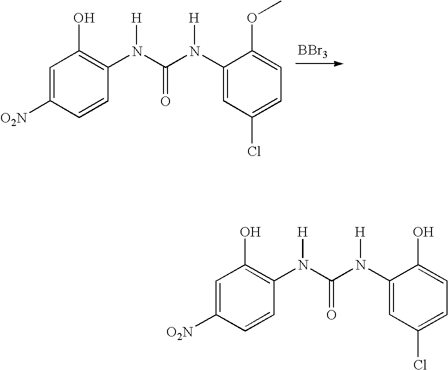

1 g of N-(3-chloro-6-methoxy-phenyl)-N′-(2-hydroxy-4-nitro-phenyl) (1.1 g; 3 mmol) was suspended in 25 mL of dichloromethane under a nitrogen atmosphere. The suspension was cooled to 0° C., added boron tribromide (4.5 mmol), stirred at 0° C. for 90 minutes and poured into water. The mixture was extracted with ethyl acetate and the organic phases were evaporated as an oil. The residue was purified by column chromatography. Yield 27%. Mp. 193-194° C.

### Example 5

### N-(2-Amino-6-hydroxy-phenyl)-N′-(3-trifluoromethyl-phenyl)urea (Compound 5A)

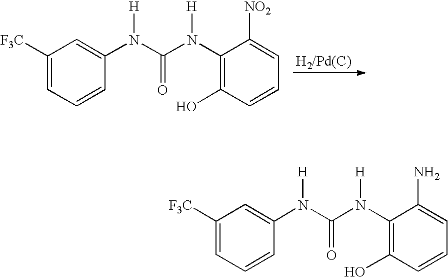

1 g of N-(2-hydroxy-6-nitro-phenyl)-N′-(3-trifluoromethyl-phenyl)urea in 100 mL of ethanol was added 0.3 g palladium on charcoal (5%). The mixture was stirred heavily under a hydrogen atmosphere for 1 hour, filtrated and evaporated. The residue was dissolved in ethyl acetate and added 5 mL of hydrogen chloride in ether (2 M), the precipitate was isolated by filtration and dried under a heat lamp. Yield 91%. Mp. 188-189° C.

### Example 6

### N-{4,5-Dichloro-2-[3-(2-chloro-5-trifluoromethyl-phenyl)-ureido]-phenyl}-acetamide (Compound 6A)

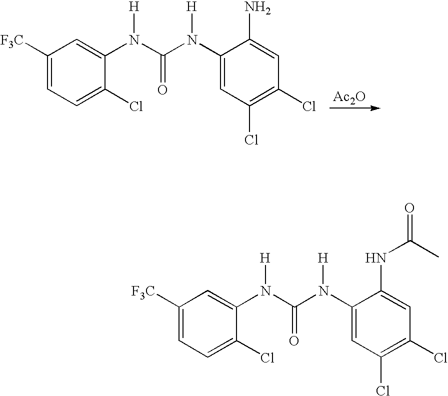

0.2 g of N-(2-amino-4,5-dichloro-phenyl)-N′-(2-chloro-5-trifluoromethyl-phenyl) urea in 15 mL of acetic acid was added 48 μL acetic acid anhydride. The reaction mixture was stirred at room temperature for 48 hours and added water. The precipitate was isolated by filtration, washed with water and dried under a heat lamp. Yield 64%. Mp. 262-264° C.

### Example 7

Biological Activity

The Morris Water Maze (MWM) is the favored test in behavioral neuroscience for the study of hippocampal-dependent learning and memory (see Morris R G: Developments of a Water Maze procedure for studying spatial learning in the rat; J. Neurosci. Meth. 1984 11 47-60). It is a standardized behavioral task to test spatial navigation in rodents, and is a highly sensitive test to assess cognition in animals.

In the MWM performance animals have to remember the position of a submerged platform (10×10 cm) within a circular (Ø 150 cm) water tank using visual spatial cues. A number of eight (n=8) male Wistar rats were used for each group in each experiment. In four trials per day, for four consecutive acquisition days, they are released from different starting points close to the wall of the tank to find their way to the submerged platform. Spatial learning abilty is evaluated by latency to locate the platform.

Scopolamine, a reference muscarinic antagonist, (s.c. administration 30 minutes prior to test start) significantly impairs spatial reference memory as measured in MWM. This learning impairment was reversed by 30 mg/kg of Compound 1 of the invention (i.p. administration 30 minutes prior to test start).

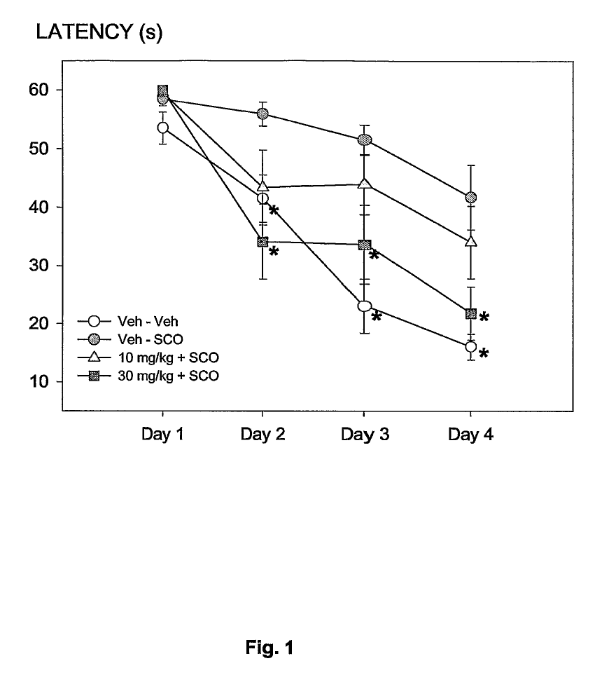

The results of this experiment are presented in FIG. 1.

1. A urea derivative represented by Formula I 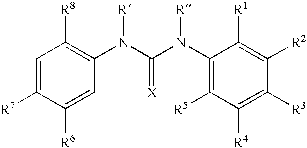
any of its enantiomers or any mixture of its enantiomers, or a prodrug, or a pharmaceutically-acceptable addition salt thereof, wherein
X represents O, S or NR′″; wherein R′″ represents hydrogen, alkyl, cycloalkyl, cycloalkyl-alkyl or cyano;
R′ and R″, independently of each other, represent hydrogen, alkyl, cycloalkyl or cycloalkyl-alkyl;
R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein
R″″ and R′″″, independently of each other, represent hydrogen, alkyl, cycloalkyl, cycloalkyl-alkyl, alkenyl, phenyl, or benzyl; and
Ar represents an aryl group or an aromatic mono- or polycyclic heterocyclic group; or
R¹ represents a group of formula —CON R″″R′″″ or —SO₂—NR″″R′″″, wherein
R″″ and R′″″, independently of each other, represent hydrogen, alkyl, cycloalkyl, cycloalkyl-alkyl, alkenyl, phenyl, or benzyl; or
R″″ and R′″″ together with the nitrogen atom to which they are attached form a heterocyclic ring; or
R¹ represents a group of formula 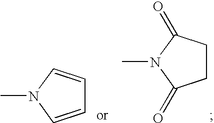
R² represents hydrogen, hydroxy, halo, haloalkyl, haloalkoxy, cyano, nitro or amino;
R³ represents hydrogen, hydroxy, halo, haloalkyl, haloalkoxy, cyano, nitro or amino;
R⁴ represents hydrogen, alkyl, hydroxy, halo, haloalkyl, haloalkoxy, cyano, nitro or amino;
R⁵ represents hydrogen, hydroxy, halo, haloalkyl, haloalkoxy, cyano, nitro or amino;
R⁶ represents hydrogen, hydroxy, halo, haloalkyl, haloalkoxy, cyano, nitro, amino or phenyl;
R⁷ represents hydrogen, hydroxy, halo, haloalkyl, haloalkoxy, cyano, nitro, amino or phenyl; and
R⁸ represents hydrogen, hydroxy, halo, haloalkyl, haloalkoxy, alkoxy, cyano, nitro or amino.

2. The urea derivative according to claim 1, wherein X represents O, S or NR′″; wherein R′″ represents hydrogen, alkyl, cycloalkyl, cycloalkyl-alkyl or cyano.

3. The urea derivative according to claim 1, wherein R′ and R″, independently of each other, represent hydrogen, alkyl, cycloalkyl or cycloalkyl-alkyl.

4. The urea derivative according to claim 3, wherein both of R′ and R″ represent hydrogen.

5. The urea derivative according to claim 1, wherein R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein
R″″ and R′″″, independently of each other, represent hydrogen, alkyl, cycloalkyl, cycloalkyl-alkyl, alkenyl, phenyl or benzyl; and
Ar represents an aryl group or an aromatic mono- or polycyclic heterocyclic group; or
R¹ represents a group of formula —CONR″″R′″″ or —SO₂—NR″″R′″″, wherein
R″″ and R′″″, independently of each other, represent hydrogen, alkyl, cycloalkyl, cycloalkyl-alkyl, alkenyl, phenyl or benzyl; or
R″″ and R′″″ together with the nitrogen atom to which they are attached form a heterocyclic ring; or
R¹ represents a group of formula

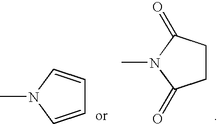

6. The urea derivative according to claim 5, wherein R¹ represents hydrogen, alkyl, hydroxy, alkoxy, halo, haloalkyl, haloalkoxy, cyano, nitro, amino, or a group of formula —NR″″(CO)R′″″, —NR″″(CO)Ar, —NR″″(CO)—NR″″R′″″, —NR″″(CO)NR′″″Ar, —NR″″(CO)CH═CH—R′″″, —NR″″(SO₂)R′″″ or —NR″″(SO₂)Ar; wherein
R″″ and R′″″, independently of each other, represent hydrogen, alkyl, cycloalkyl, cycloalkyl-alkyl, alkenyl, phenyl or benzyl; and
Ar represents phenyl, pyrrolyl, imidazolyl, pyrazolyl or pyridinyl; or
R¹ represents a group of formula —CONR″″R′″″ or —SO₂—NR″″R′″″, wherein
R″″ and R′″″, independently of each other, represent hydrogen, alkyl, cycloalkyl, cycloalkyl-alkyl, alkenyl, phenyl or benzyl; or
R″″ and R′″″ together with the nitrogen atom to which they are attached form a heterocyclic ring selected from pyrrolidinyl, piperidinyl, morpholinyl and piperazinyl.

7. The urea derivative according to claim 6, wherein R¹ represents hydrogen, alkyl, hydroxy, alkoxy, amino, or a group of formula —NR″″(CO)R′″″; wherein
R″″ and R′″″, independently of each other, represent hydrogen, alkyl, cycloalkyl, cycloalkyl-alkyl, alkenyl, phenyl or benzyl; or
R¹ represents a group of formula

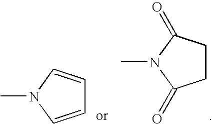

8. The urea derivative according to claim 6, wherein R¹ represents hydrogen, alkyl, hydroxy, alkoxy, amino, or —NH(CO)alkyl.

9. The urea derivative according to claim 1, wherein R² represents hydrogen, hydroxy, halo, haloalkyl, haloalkoxy, cyano, nitro or amino.

10. The urea derivative according to claim 9, wherein R² represents hydrogen, hydroxy or halo.

11. The urea derivative according to claim 1, wherein R³ represents hydrogen, hydroxy, halo, haloalkyl, haloalkoxy, cyano, nitro or amino.

12. The urea derivative according to claim 11, wherein R³ represents hydrogen, hydroxy, halo or nitro.

13. The urea derivative according to claim 1, wherein R⁴ represents hydrogen, alkyl, hydroxy, halo, haloalkyl, haloalkoxy, cyano, nitro or amino.

14. The urea derivative according to claim 13, wherein R⁴ represents hydrogen, alkyl or halo.

15. The urea derivative according to claim 1, wherein R⁵ represents hydrogen, hydroxy, halo, haloalkyl, haloalkoxy, cyano, nitro or amino.

16. The urea derivative according to claim 15, wherein R⁵ represents hydrogen, nitro or amino.

17. The urea derivative according to claim 1, wherein R⁶ represents hydrogen, hydroxy, halo, haloalkyl, haloalkoxy, cyano, nitro, amino or phenyl.

18. The urea derivative according to claim 17, wherein R⁶ represents hydrogen, halo, haloalkyl or phenyl.

19. The urea derivative according to claim 1, wherein R⁷ represents hydrogen, hydroxy, halo, haloalkyl, haloalkoxy, cyano, nitro, amino or phenyl.

20. The urea derivative according to claim 19, wherein R⁷ represents hydrogen, nitro or phenyl.

21. The urea derivative according to claim 1, wherein R⁸ represents hydrogen, hydroxy, halo, haloalkyl, haloalkoxy, alkoxy, cyano, nitro or amino.

22. The urea derivative according to claim 21, wherein R⁸ represents hydrogen, hydroxy, halo or alkoxy.

23. The urea derivative according to claim 1, wherein
R¹ represents hydrogen, alkyl, hydroxy, alkoxy, amino or —NH(CO)methyl;
R² represents hydrogen, hydroxy or halo;
R³ represents hydrogen, hydroxy, halo or nitro;
R⁴ represents hydrogen, alkyl or halo;
R⁵ represents hydrogen, alkyl, amino or nitro;
R⁶ represents hydrogen, halo, haloalkyl or phenyl;
R⁷ represents hydrogen or phenyl; and
R⁸ represents hydrogen, hydroxy, halo or alkoxy.

24. The urea derivative according to claim 1, wherein
R¹ represents hydroxy;
R² represents hydrogen or halo;
R³ represents hydrogen or nitro;
R⁴ represents hydrogen or halo;
R⁵ represents hydrogen, nitro or amino;
R⁶ represents halo or haloalkyl;
R⁷ represents hydrogen or phenyl; and
R⁸ represents hydrogen, halo or alkoxy.

25. The urea derivative according to claim 1, wherein
R¹ represents hydrogen;
R² represents hydrogen, hydroxy or halo (chloro);
R³ represents hydrogen or hydroxy;
R⁴ represents alkyl or halo;
R⁵ represents hydrogen;
R⁶ represents hydrogen, haloalkyl or phenyl;
R⁷ represents hydrogen or phenyl; and
R⁸ represents hydrogen or halo.

26. The urea derivative according to claim 1, wherein
R¹ represents alkyl, alkoxy, amino or N-alkylcarbonyl-amino;
R² represents hydrogen;
R³ represents hydroxy or halo;
R⁴ represents hydrogen or halo;
R⁵ represents hydrogen;
R⁶ represents haloalkyl;
R⁷ represents hydrogen; and
R⁸ represents hydrogen or halo.

27. The urea derivative of claim 23, which is
N-(3-Chloro-6-hydroxy-phenyl)-N′-(2-chloro-5-trifluoromethyl-phenyl)-urea;
N-(2-Amino-6-hydroxy-phenyl)-N′-(3-trifluoromethyl-phenyl)-urea;
N-(5-Chloro-2-hydroxy-phenyl)-N′-(2-hydroxy-4-nitro-phenyl)-urea;
N-(2-Amino-4,5-dichloro-phenyl)-N′-(2-chloro-5-trifluoromethyl-phenyl)-urea;
N-{4,5-Dichloro-2-[3-(2-chloro-5-trifluoromethyl-phenyl)-ureido]phenyl}-acetamide;
N-(3-Chloro-4-hydroxy-phenyl)-N′-(3-trifluoromethyl-phenyl)-urea;
N-(4-Hydroxy-6-methyl-phenyl)-N′-(3-trifluoromethyl-phenyl)-urea;
N-(3,5-Dichloro-4-hydroxy-phenyl)-N′-(3-trifluoromethyl-phenyl)urea;
N-(2-Chloro-5-trifluoromethyl-phenyl)-N′-(3,5-dichloro-4-hydroxy-phenyl)urea;
N-(Biphenyl-3-yl)-N′-(3,5-dichloro-4-hydroxy-phenyl)urea;
N-(Biphenyl-4-yl)-N′-(3,5-dichloro-4-hydroxy-phenyl)urea;
N-(Biphenyl-4-yl)-N′-(5-chloro-2-hydroxy-phenyl)urea;
N-(3,5-Dichloro-2-hydroxy-phenyl)-N′-(2-chloro-5-trifluoromethyl-phenyl)-urea;
N-(3-Bromo-5-chloro-2-hydroxy-phenyl)-N′-(2-chloro-5-trifluoromethyl-phenyl)-urea;
N-(2-Chloro-5-trifluoromethyl-phenyl)-N′-(3-hydroxy-5-methyl-phenyl)urea;
N-(3-Hydroxy-5-methyl-phenyl)-N′-(3-trifluoromethyl-phenyl)urea;
N-(2-Chloro-5-trifluoromethyl-phenyl)-N′-(4-hydroxy-2-methyl-phenyl)urea;
N-(5-Chloro-2-methoxy-phenyl)-N′-(2-chloro-5-trifluoromethyl-phenyl)urea;
N-(2-Hydroxy-6-nitro-phenyl)-N′-(3-trifluoromethyl-phenyl)urea; or
N-(3-Chloro-6-methoxy-phenyl)-N′-(2-hydroxy-4-nitro-phenyl)urea;
or an enantiomer or a mixture of its enantiomers, or a pharmaceutically-acceptable addition salt thereof.

28. A pharmaceutical composition comprising a therapeutically effective amount of a urea derivative of claim 1, or a pharmaceutically-acceptable addition salt thereof, together with at least one pharmaceutically-acceptable carrier or diluent.

29. A method of treatment, prevention or alleviation of a disease or a disorder or a condition of a living animal body, including a human, which disorder, disease or condition is responsive to modulation of nicotinic acetylcholine α7 receptors, which method comprises the step of administering to such a living animal body in need thereof a therapeutically effective amount of a urea derivative of claim 1.

30. The method according to claim 29, wherein the disease, disorder or condition relates to the central nervous system.

31. The method according to claim 30, wherein the disease, disorder or condition is anxiety, cognitive disorders, learning deficit, memory deficits and dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, schizophrenia, obsessive compulsive disorders (OCD), panic disorders, eating disorders such as anorexia nervosa, bulimia and obesity, narcolepsy, nociception, AIDS-dementia, senile dementia, periferic neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania and jet-lag.

32. The method according to claim 29, wherein the disease, disorder or condition are associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

33. The method according to claim 29, wherein the disease, disorder or condition is related to the endocrine system, such as thyrotoxicosis, pheochromocytoma, hypertension and arrhythmias.

34. The method according to claim 29, wherein the disease, disorder or condition is a neurodegenerative disorders, including transient anoxia and induced neuro-degeneration.

35. The method according to claim 29, wherein the disease, disorder or condition is an inflammatory disorder, including inflammatory skin disorders such as acne and rosacea, Chron's disease, inflammatory bowel disease, ulcerative colitis and diarrhoea.

36. The method according to claim 29, wherein the disease, disorder or condition is mild, moderate or even severe pain of acute, chronic or recurrent character, as well as neuropathic pain and pain caused by migraine, postoperative pain, phantom limb pain, neuropathic pain, chronic headache, central pain, pain related to diabetic neuropathy, to post therapeutic neuralgia, or to peripheral nerve injury.

37. The method according to claim 29, wherein the disease, disorder or condition is associated with withdrawal symptoms caused by termination of use of addictive substances, including nicotine containing products such as tobacco, opioids such as heroin, cocaine and morphine, benzodiazepines and benzodiazepine-like drugs and alcohol.

38. (canceled)
